(19) United States
(12) Patent Application Publication
(10) Pub. No.: US 2013/0089829 A1
(43) Pub. Date: Apr. 11, 2013

(54) Light Diluting Toothbrush Bristles

(71) Applicant: Biolase, Inc., Irvine, CA (US)

(72) Inventors: Dmitri Boutoussov, Dana Point, CA (US); William E. Brown, JR., Roswell, GA (US); Matthew F. Duncan, San Clemente, CA (US)

(73) Assignee: BIOLASE, INC., Irvine, CA (US)

(21) Appl. No.: 13/646,044

(22) Filed: Oct. 5, 2012

Related U.S. Application Data
(60) Provisional application No. 61/545,005, filed on Oct. 7, 2011.

(51) Int. Cl. A61N 5/06; A46B 9/04

(52) U.S. Cl. 433/29

(57) ABSTRACT

A tissue treatment device is disclosed. A tissue treatment device includes a bristle having a longitudinal axis, where the bristle is configured to emit a peak concentration of electromagnetic radiation along a direction that is not parallel to the longitudinal axis. The tissue treatment device also includes a brush head for holding the bristle, where the bristle projects outwardly from the brush head and is configured to brush a surface. The tissue treatment device further includes an electromagnetic energy source optically coupled to the bristle. The bristle is a waveguide that is configured to receive electromagnetic radiation from the electromagnetic energy source at a coupling point and to emit the received electromagnetic radiation at a tip.

### CROSS-REFERENCE TO RELATED APPLICATIONS

This application claims priority to U.S. Provisional Patent Application No. 61/545,005, filed Oct. 7, 2011, entitled “Light Diluting Toothbrush Bristles,” which is herein incorporated by reference in its entirety.

### TECHNICAL FIELD

The technology described herein relates generally to a tissue treatment device and more particularly to a tissue treatment device including a light-guiding bristle and an electromagnetic energy source.

### BACKGROUND

Prevention of tooth decay is important for good health. Tooth decay results from the growth of bacteria on the tooth, where the bacteria may grow on the tooth as plaque. Treating and preventing tooth decay may involve removal of the plaque via mechanical cleaning techniques. However, the treatment and prevention of tooth decay using mechanical cleaning techniques has certain limitations. For example, a toothbrush or dental floss often cannot penetrate into skin tissue or into deep pockets between teeth and gums to remove bacterial and viral contamination. Further, toothpaste is largely ineffective in destroying bacteria and viruses. Electromagnetic radiation (e.g., output from a laser or laser diode) can provide a more effective tool for dental cleaning. For example, a toothbrush with a laser source can direct low power electromagnetic radiation onto teeth, gums, and other areas of the mouth to remove bacteria and viruses. In addition, electromagnetic radiation may be applied on teeth for whitening purposes. For example, a whitening agent may be applied to teeth, and then a low power laser may be directed at the teeth to activate the agent to facilitate teeth whitening.

### SUMMARY

A tissue treatment device is disclosed. A tissue treatment device includes a bristle having a longitudinal axis, where the bristle is configured to emit a peak concentration of electromagnetic radiation along a direction that is not parallel to the longitudinal axis. The tissue treatment device also includes a brush head for holding the bristle, where the bristle projects outwardly from the brush head and is configured to brush a surface. The tissue treatment device further includes an electromagnetic energy source optically coupled to the bristle. The bristle is a waveguide that is configured to receive electromagnetic radiation from the electromagnetic energy source at a coupling point and to emit the received electromagnetic radiation at a tip.

Another tissue treatment device includes an electromagnetic energy source configured to generate electromagnetic radiation of a wavelength. An optical lens is configured to focus the electromagnetic radiation of the wavelength at a focal point that is a distance from the optical lens. The tissue treatment device also includes a plurality of light-shielding bristles having lengths that are longer than the distance, where the light-shielding bristles are substantially opaque to the electromagnetic radiation of the wavelength. The tissue treatment device further includes a brush head for holding the lens and the light-shielding bristles. The light-shielding bristles surround the optical lens on the brush head and project outwardly from the brush head in a direction substantially perpendicular to the brush head.

Another tissue treatment device includes a first bristle configured to emit electromagnetic radiation of a first wavelength, and a second bristle configured to emit electromagnetic radiation of a second wavelength. The oral treatment device also includes a brush head for holding the first and the second bristles. The bristles project outwardly from the brush head and are configured to brush a surface. The oral treatment device further includes an electromagnetic energy source optically coupled to the first and the second bristles, where the electromagnetic energy source is configured to generate the electromagnetic radiation of the first and the second wavelengths. The first and the second bristles are configured to act as waveguides for the electromagnetic radiation of the first and the second wavelengths, respectively.

### BRIEF DESCRIPTION OF THE FIGURES

FIG. 1 depicts an example tissue treatment device that utilizes emitted electromagnetic radiation to treat a surface.

FIG. 2 illustrates example light-emitting bristles having modified output ends configured to emit electromagnetic radiation in a wide-angle ring pattern and a pattern having opposing lobes.

FIG. 3 illustrates example light-emitting bristles having modified coupling points.

FIG. 4 illustrates example brush heads having a plurality of bristles with beveling on a single side.

FIG. 5 illustrates a use of bristles having beveling on a single side to treat a surface of a tooth.

FIG. 6 illustrates bristles having tips with surface roughness features.

FIG. 7 illustrates aspects of example tissue treatment devices utilizing light-shielding bristles.

FIG. 8 depicts example oral treatment devices configured to utilize multiple wavelengths of light.

### DETAILED DESCRIPTION

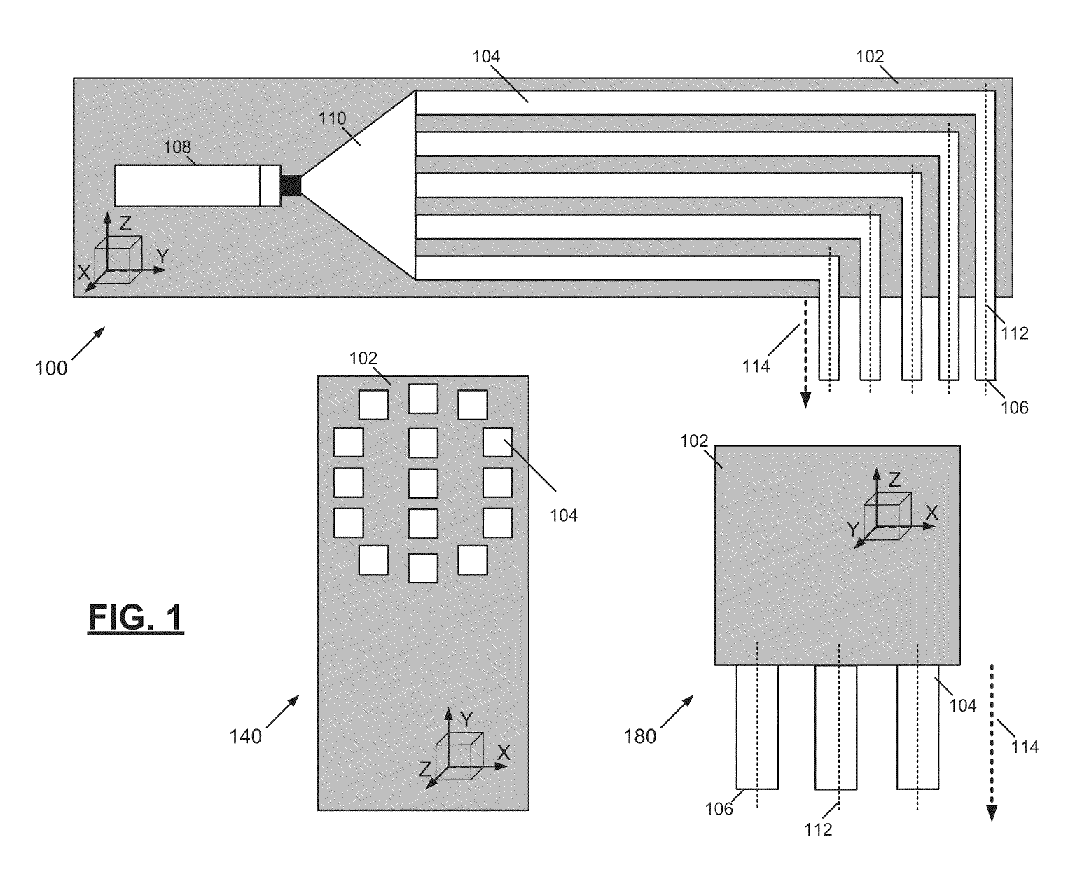

FIG. 1 depicts an example tissue treatment device that utilizes emitted electromagnetic radiation to treat a surface. A cross-sectional view of the treatment device 100 illustrates basic components of the tissue treatment device. The tissue treatment device includes a plurality of bristles 104 held by a brush head 102. Each of the bristles 104 has a longitudinal axis 112 substantially parallel to a direction 114 that is perpendicular to the brush head 102. The bristles 104 are optically-transmissive bristles that function as waveguides configured to receive electromagnetic radiation at a coupling point 110, to guide the electromagnetic radiation along a length of the bristle 104, and to emit the electromagnetic radiation at a tip 106. The bristles 104 are further configured to emit a peak concentration of the electromagnetic radiation along a direction that is not parallel to the longitudinal axes 112. The emission of the peak concentration of the electromagnetic radiation along the direction that is not parallel to the longitudinal axes 112 may provide a safety feature (i.e., reducing power density near the tip 106 to lessen a probability of causing harm to a human eye) or may be used for efficient tissue treatment (i.e., by directing the electromagnetic radiation to target surfaces for treatment). The bristles 104 project outwardly from the brush head 102 and are used to scour or brush a surface (e.g., teeth, gums of a mouth). In an example, the bristles 104 are made of fiber optic cables and tips that are configured to emit the electromagnetic radiation and to brush the surface.

The cross-sectional view of the tissue treatment device 100 further illustrates an electromagnetic energy source 108 of the device. The electromagnetic energy source 108 is optically coupled to the plurality of the bristles 104 at the coupling point 110 and is used to generate the electromagnetic radiation that is emitted at the tips 106 of the bristles 104. The electromagnetic energy source 108 can use any suitable means of generating the electromagnetic radiation and may be a lamp, semiconductor laser, laser diode, or a variety of other light-emitting devices. In one example, the electromagnetic energy source 108 utilizes a plurality of light sources, including a first source adapted to be used in a teeth whitening procedure (e.g., a laser or laser diode capable of producing light of a wavelength range of approximately 390 nm-480 nm) and a second source adapted to be used in a gum treatment procedure (e.g., a laser or laser diode capable of producing light of a wavelength range of approximately 620 nm-680 nm). The optical coupling 110 between the electromagnetic energy source 108 and the bristles 104 can utilize any suitable method of coupling the electromagnetic radiation from the energy source 108 to the bristles 104 and may employ various lenses, collimators, and adapters to reduce coupling loss between the source 108 and the bristles 104.

A second view of the tissue treatment device 140 illustrates an example arrangement of the bristles 104 on the brush head 102. In different examples, the bristles 104 are arranged on the brush head 102 in a variety of different patterns (e.g., circular patterns, rectangular patterns including rows and columns of bristles 104). In one example, the bristles 104 are arranged around a periphery of the brush head 102, and the bristles 104 are each configured to direct the emitted electromagnetic radiation in a direction that is substantially perpendicular to the longitudinal axes 112 of the bristles and towards a center area of the brush head 102. In another example, the brush head 102 is an oscillating brush head configured to rotate along an axis that is parallel to the direction 114 that is perpendicular to the brush head 102.

A third view of the treatment device 180 illustrates a view of the brush head 102 from a different angle and depicts the bristles 104 projecting outwardly from the brush head 102. The bristles 104 project outwardly from the brush head 102 at an angle that is approximately parallel to the direction 114 that is perpendicular to the brush head 102.

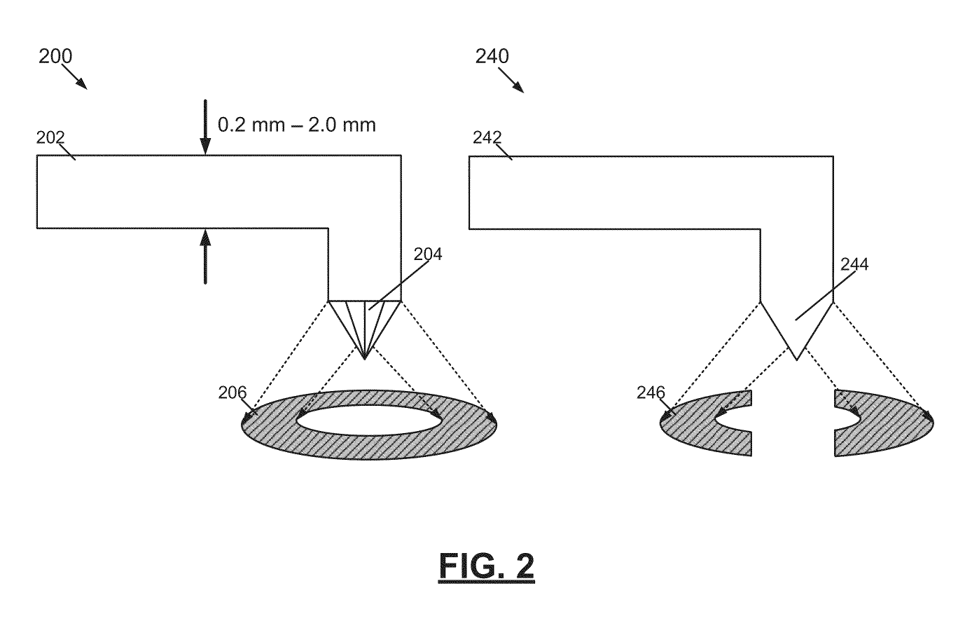

FIG. 2 illustrates example light-emitting bristles 202, 242 having modified output ends configured to emit electromagnetic radiation in a wide-angle ring pattern 206 and a pattern having opposing lobes 246. At 200, the light-emitting bristle 202 includes a tip 204 having a cone shape. The bristle 202 has a diameter within a range of approximately 0.2 mm to 2.0 mm and may be made of various different materials (e.g., polyethylene, polycarbonate, glass, sapphire, quartz, hollow waveguide, liquid core, quartz silica, germanium oxide). As illustrated at 200, the tip 204 having the cone shape is configured to emit the electromagnetic radiation in the wide-angle ring pattern 206. A diameter of the ring pattern 206 increases as a distance from the tip 204 increases (i.e., the electromagnetic radiation disperses as the distance from the tip 204 increases). By modifying a geometry of the conical tip 204, properties of the ring pattern 206 (e.g., diameter of inner and outer rings, angle of the ring pattern 206 relative to the tip 204) may be altered.

At 240, the light-emitting bristle 242 includes a tip 244 having a double-beveled shape. The tip 244 having the double-beveled shape is configured to emit the electromagnetic radiation in the pattern having opposing lobes 246, where a size of the opposing lobes 246 increases as a distance from the tip 244 increases. By modifying geometry of the double-beveled tip 244, properties of the opposing lobes 246 may be altered.

The bristles 202, 242 having the conical tip 204 and the double-beveled tip 244, respectively, may be used in the context of the tissue treatment device of FIG. 1 to emit the electromagnetic radiation along a direction that is not parallel to a longitudinal axis of the bristle 202, 242. Both tips 204, 244 differ from forward-firing tips, which are configured to produce a more collimated beam of light that is substantially parallel to a longitudinal axis of the tip.

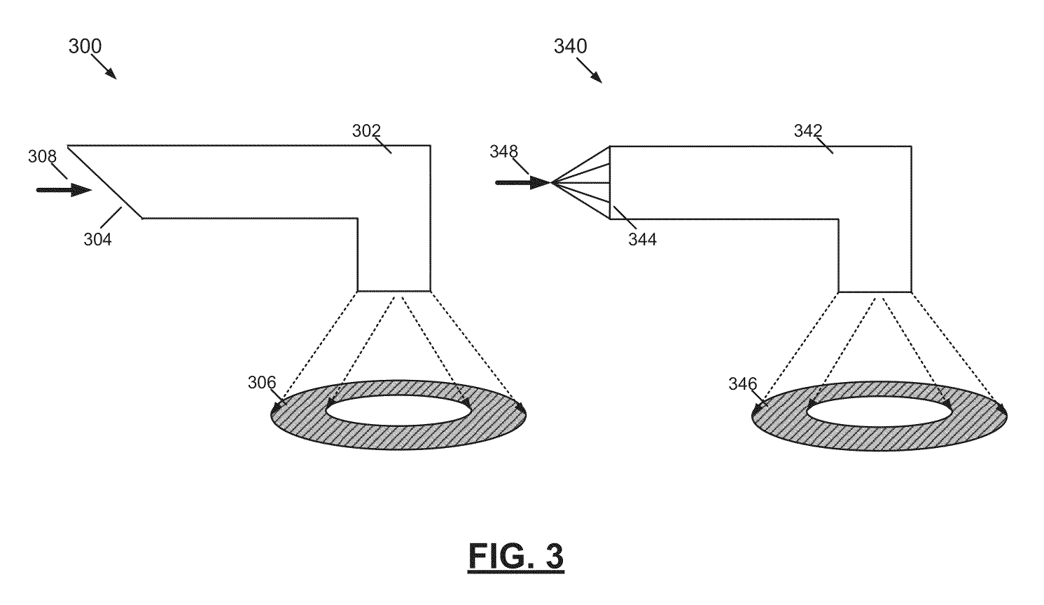

FIG. 3 illustrates example light-emitting bristles 302, 342 having modified coupling points 304, 344. At 300, the light-emitting bristle 302 includes the coupling point 304 configured to receive electromagnetic radiation 308 from an electromagnetic source. The coupling point 304 has a single beveled edge, and the beveled shape of the coupling point 304 causes the bristle 302 to emit the electromagnetic radiation 308 in a wide-angle ring pattern 306. A diameter of the ring pattern 306 increases as a distance from the bristle 302 increases (i.e., the electromagnetic radiation 308 disperses as the distance from the bristle 302 increases). Similarly, at 340, the light emitting-bristle 342 includes the coupling point 344 configured to receive electromagnetic radiation 348 from an electromagnetic source. The coupling point 344 has a cone shape or a double-beveled edge. The cone shape or the double-beveled edge of the coupling point 344 causes the bristle 342 to emit the electromagnetic radiation 348 in a wide-angle ring pattern 346. The bristles 302, 342 having the modified coupling points 304, 344 may be used in the context of the tissue treatment device of FIG. 1 to emit the electromagnetic radiation 308, 348 along a direction that is not parallel to a longitudinal axis of the bristle 302, 342.

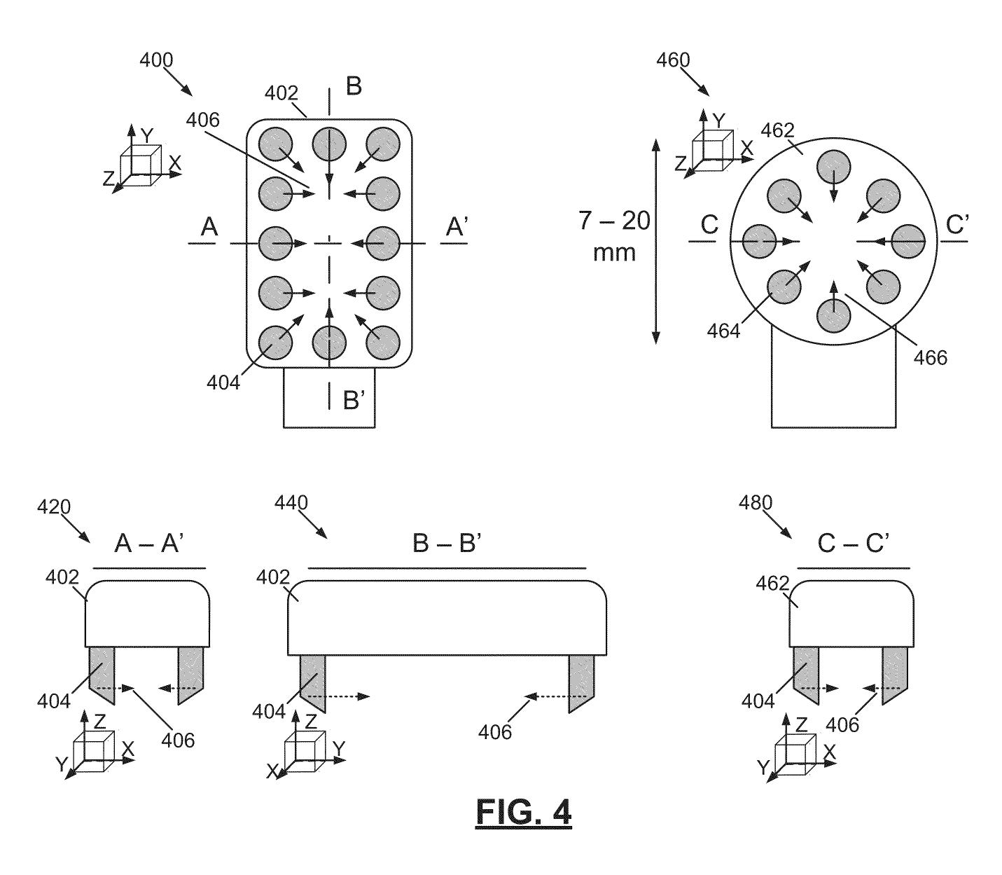

FIG. 4 illustrates example brush heads 402, 462 having a plurality of bristles 404, 464 with beveling on a single side. A first view 400 illustrates a tissue treatment device with the brush head 402 holding the plurality of bristles 404, where the bristles 404 are configured to emit electromagnetic radiation 406 towards a center area of the brush head 402. As illustrated in views 420 and 440 of FIG. 4, the electromagnetic radiation 406 is directed towards the center area of the brush head 402 due to tips of the bristles 404 having beveling on a single side. The electromagnetic radiation 406 emitted in this direction may provide eye safety by directing the electromagnetic radiation inward and towards a center area of the brush head 402, rather than outward (i.e., in a Z direction), where it could affect or harm a human eye. Further, the emission of the electromagnetic radiation 406 in this direction may allow for an efficient power delivery of electromagnetic radiation 406 to a surface that is being brushed by the bristles 404 (i.e., a tooth surface).

Views 420 and 440 illustrate cross-sectional planes of the tissue treatment device having the bristles 404 with beveling on the single side. With reference to the first view 400 of FIG. 4, the view 420 illustrates the A-A′ cross-section, along an X direction. As illustrated at 420, the bristles 404 have tips with the beveling on the single side, which causes the electromagnetic radiation 406 emitted from the tips to be directed towards the center area of the brush head 402 and in a direction that is substantially perpendicular to a Z direction. Similarly, the view 440 illustrates the B-B′ cross-section, along a Y direction, with the bristles 404 having tips with the beveling on the single side to cause the electromagnetic radiation 406 emitted by the tips to be directed towards the center area of the brush head 402 and in a direction that is substantially perpendicular to the Z direction.

At 460, a second brush head 462 and bristle arrangement is depicted. On the brush head 462, the bristles 464 are arranged in a circular pattern. The bristles 464 have tips with beveling on a single side, which causes electromagnetic radiation 466 emitted by the tips to be directed inward, towards a center area of the brush head 462. View 480 illustrates cross-sectional plane C-C′ of the brush head 462, along an X direction. As illustrated in the view 480, the tips of the bristles 464 have beveling on a single side, and the emitted electromagnetic radiation 406 is directed towards the center area of the brush head 462 and in a direction that is substantially perpendicular to the Z direction. In the example of FIG. 4, the brush head 462 has a circular shape with a diameter in a range of approximately 7 mm-20 mm. The example shapes of the brush heads 402, 462 and the arrangement of the bristles 404, 464 depicted in FIG. 4 are exemplary only, and a variety of other shapes and bristle arrangements may be used in other examples.

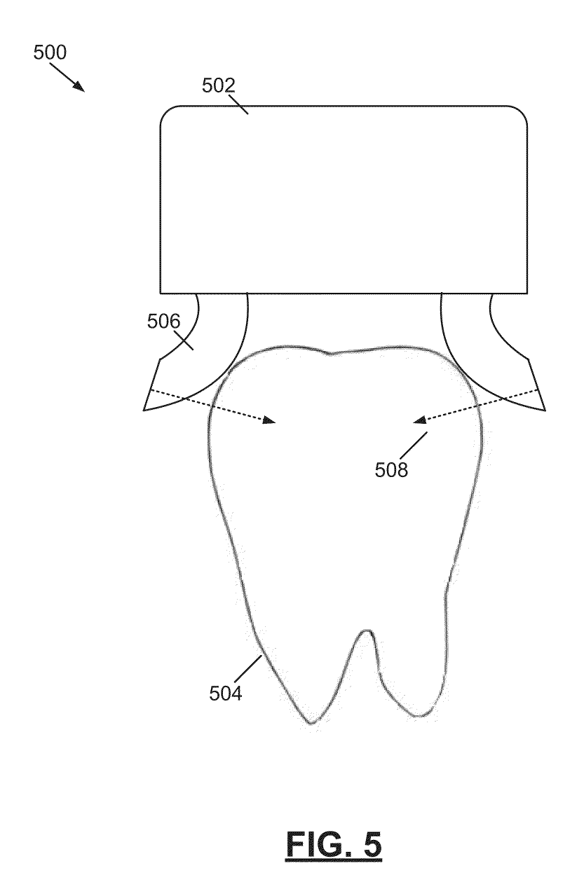

FIG. 5 illustrates a use of bristles 506 having beveling on a single side to treat a surface of a tooth 504. As explained above with respect to FIG. 4, tissue treatment devices utilizing bristles with beveling on a single side may be configured to emit electromagnetic radiation towards a center area of a brush head. The emission of the electromagnetic radiation in this direction may allow for an efficient power delivery of electromagnetic radiation to a surface that is being brushed by the bristles. This efficient power delivery is illustrated in the system 500 of FIG. 5. In FIG. 5, a brush head 502 includes the bristles 506 having beveling on the single side. The beveling on the single side causes the electromagnetic radiation 508 emitted by the bristles 506 to be towards a center area of the brush head 502 and substantially perpendicular to a direction that is parallel to longitudinal axes of the bristles 506. As illustrated at 500, a tooth 504 compresses the bristles 506, causing a surface of the tooth 504 to be in a volume receiving a peak concentration of the inwardly-directed electromagnetic radiation 508.

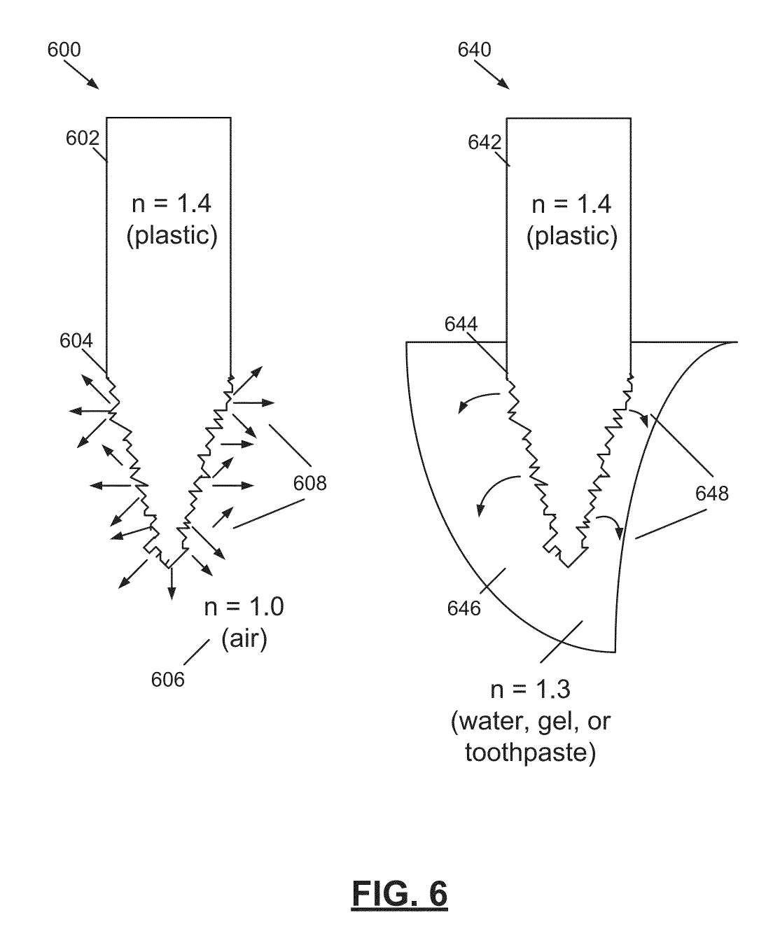

FIG. 6 illustrates bristles 602, 642 having tips 604, 644 with surface roughness features. At 600, the bristle 602 has an index of refraction of approximately 1.4 and is surrounded by air 606 having an index of refraction of approximately 1.0. The bristle 602 may be made of plastic or another optically-transmissive material with an index of refraction of approximately 1.4. When in the air 606 environment, electromagnetic radiation 608 emitted from the tip 604 of the bristle 602 is scattered in a plurality of directions by the surface roughness features of the tip 604. The tip 604 with the surface roughness features may have a “glowing” appearance, owing to the scattering of the electromagnetic radiation 608 in the plurality of directions. The glowing tip 604 does not produce a focused beam of light having a power concentration high enough to affect or harm a human eye, and thus, the surface roughened tip 604 may be a safety feature that may be implemented in the context of the example tissue treatment device of FIG. 1. The surface roughness features of the tip 604 may have sizes within a range of approximately 5 μm to 50 μm. The surface roughness features of the tip 604 may be generated by an injection molding process, a sandblasting process, a beadblasting process, or another process configured to roughen the tip 604 of the bristle 602.

At 640, the bristle 642 has an index of refraction of approximately 1.4 and may be made of plastic or another optically-transmissive material having this approximate index of refraction. Like the bristle 602 illustrated at 600, the bristle 642 has a tip with surface roughness features (e.g., surface roughness features sized within a range of approximately 5 μm to 50 μm). By contrast to the bristle 602, the tip 644 of the bristle 642 is not surrounded in an air environment on all sides. Rather, the tip 644 is in contact with a substance 646 having an index of refraction of approximately 1.3. The substance 646 in contact with the tip 644 may be a water, gel, toothpaste (e.g., a non-foaming, viscous toothpaste), or another substance that is contained on a surface (e.g., a peroxide-free dentifrice or a reduced-peroxide dentifrice configured to be non-foaming, a toothpaste or other dentifrice including anti-foaming agents). When the tip 644 having the surface roughness features is in contact with the substance 646, the tip 644 may transfer a substantial portion of electromagnetic radiation 648 emitted from the tip 644 to the substance 646. The transfer of the substantial portion of the electromagnetic radiation 648 from the tip 644 to the substance 646 is in contrast to the scattering of the electromagnetic radiation 608 in the plurality of directions exhibited when the surface roughened tip 604 is surrounded in air 606.

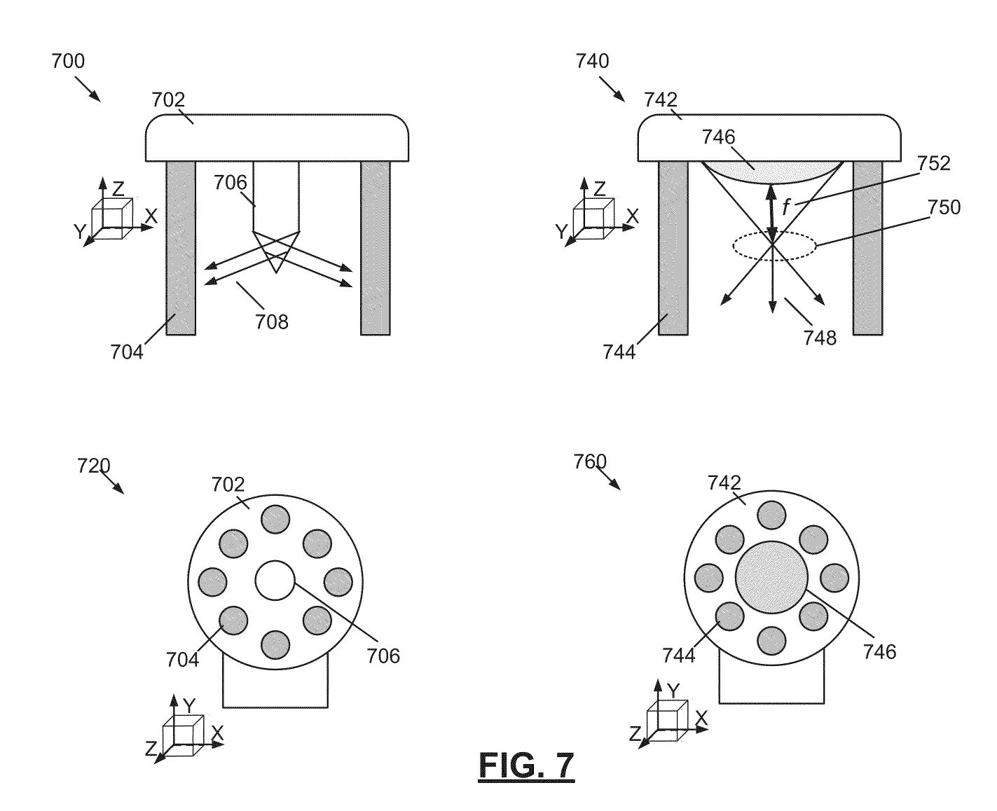

FIG. 7 illustrates aspects of example tissue treatment devices 700, 740 utilizing light-shielding bristles 704, 744. The example tissue treatment device 700 includes a brush head 702 and a light-emitting bristle 706 having a longitudinal axis, where the light-emitting bristle 706 is configured to emit a peak concentration of electromagnetic radiation 708 along a direction that is not parallel to the longitudinal axis. In the example of FIG. 7, the light-emitting bristle 706 has a tip with a shape configured to cause the emission of the electromagnetic radiation 708 along the direction that is not parallel to the longitudinal axis. Specifically, the light-emitting bristle 706 of FIG. 7 has a cone shape or a double-beveled shape, such that the emitted electromagnetic radiation 708 is emitted from the tip at an angle, as illustrated at 708.

In the tissue treatment device 700, the light-emitting bristle 706 is surrounded by a plurality of the light-shielding bristles 704. The light-shielding bristles 704 are substantially opaque to the electromagnetic radiation 708 emitted by the bristle 706, such that the electromagnetic radiation 708 emitted at the angle is effectively confined within a volume of space between the light-shielding bristles 704.

In the view 720 of the tissue treatment device 700, the light-shielding bristles 704 are located at a peripheral area of the brush head 702. The brush head 702 is circular in the example of FIG. 7, but various other shapes and geometries for the brush head 702 may be used in other examples. The brush head 702 may be an oscillating brush head configured to rotate around an axis that is parallel to the Z direction. The light-emitting bristle 706 is located near a center area of the brush head 702. Other examples include a plurality of light-emitting bristles 706 surrounded by the light-shielding bristles 704. Further, in other examples, various other tip designs are used with the light-emitting bristle 706 (e.g., a tip having beveling on a single side as illustrated in FIG. 4, a tip having surface roughness features as illustrated in FIG. 6).

The example tissue treatment device 740 of FIG. 7 includes a brush head 742 and an optical lens 746. The brush head 742 may be an oscillating brush head configured to rotate around an axis that is parallel to the Z direction. The optical lens 746 is configured to focus electromagnetic radiation 748 produced by an electromagnetic energy source at a focal point 752 located at a distance f from the lens 746. The optical lens 746 is thus configured to cause a peak concentration 750 of the electromagnetic radiation 748 to be located near the focal point 752. The optical lens 746 is surrounded on the brush head 742 by a plurality of the light-shielding bristles 744. The light-shielding bristles 744 project outwardly from the brush head 742 in a direction substantially perpendicular to a surface of the brush head 742. The light-shielding bristles 744 are substantially opaque to the electromagnetic radiation 748 transmitted by the optical lens 746. Further, the light-shielding bristles 744 have lengths that are longer than the distance f separating the optical lens 746 and the focal point 752, such that the peak concentration 750 of the electromagnetic radiation 748 is located within a volume of space between the light-shielding bristles 744. In the view 760 of the tissue treatment device 740, the light-shielding bristles 744 are located at a peripheral area of the brush head 742, and the optical lens 746 is located near a center area of the brush head 742

Although the light-shielding bristles 704, 744 of the example treatment devices 700, 740 of FIG. 7 are described as being used to confine the light emitted by other elements (i.e., the light-emitting tip 706 and the optical lens 746, respectively), in other examples, the light-shielding bristles 704, 744 may also be used to emit electromagnetic radiation. In one example, the light-shielding bristles 704, 744 may be configured to emit electromagnetic radiation of a wavelength that is different than a wavelength of the electromagnetic radiation 708, 748 emitted by the tip 706 and lens 746, respectively.

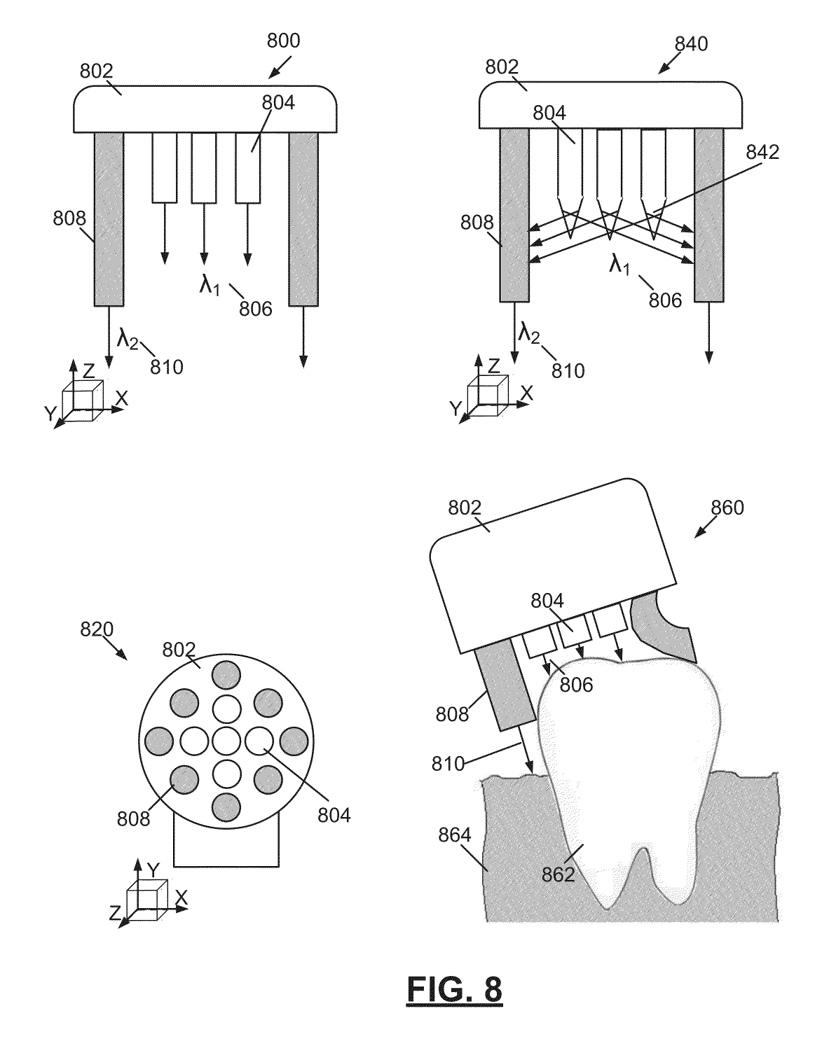

FIG. 8 depicts example oral treatment devices 800, 840 configured to utilize multiple wavelengths of light. The example oral treatment device 800 includes a first plurality of bristles 804 configured to emit electromagnetic radiation 806 at a first wavelength (λ₁), and a second plurality of bristles 808 configured to emit electromagnetic radiation 810 at a second wavelength (λ₂). The bristles 804, 808 are held by a brush head 802, and the bristles 804, 808 project outwardly from the brush head 802 and are used to brush a surface (e.g., a surface of a tooth or a surface of gums of a mouth). The bristles 804, 808 are optically coupled to an electromagnetic energy source that is configured to generate the electromagnetic radiation of the first and the second wavelengths 806, 810. The bristles 804, 808 act as waveguides configured to receive the electromagnetic radiation of the first and the second wavelengths 806, 810, respectively, and to guide the radiation before emitting it via tips of the bristles 804, 808.

The dual-wavelength oral treatment device 800 may utilize the electromagnetic radiation of the first and second wavelengths 806, 810 for performing different treatment procedures. In one example, the electromagnetic radiation of the first wavelength 806 is configured to whiten teeth, and the electromagnetic radiation of the second wavelength 810 is configured to treat gums of a mouth (e.g., killing bacteria in or on the gums). In this example, the first wavelength 806 may be within a range of approximately 390 nm to 480 nm (e.g., 480 nm blue light), and the second wavelength 810 may be within a range of approximately 620 nm to 680 nm (e.g., 655 nm light). Further, the bristles 804, 808 may be made of materials specifically designed to transmit the electromagnetic radiation 806, 810 at these wavelengths. For example, the bristles 804 used for the first wavelength 806 may be made of polyethylene or polycarbonate, and the bristles 808 used for the second wavelength 810 may be made of transparent red-tinted polyethylene (e.g., medium tint) or made of material that is transparent to the electromagnetic radiation of the second wavelength 810 and is coated with a substance that is reflective or opaque to the electromagnetic radiation of the first wavelength 806.

As illustrated in the example of FIG. 8, the bristles 804 configured to emit the electromagnetic radiation of the first wavelength 806 have a length that is less than a length of the bristles 808 configured to emit the electromagnetic radiation of the second wavelength 810. In one example, the bristles 804 have a length within a range of approximately 2 mm to 4 mm, and the bristles 808 have a length within a range of approximately 5 mm to 8 mm. Further, as illustrated in a view 820 of the oral treatment device 800, the bristles 804 configured to emit the electromagnetic radiation of the first wavelength 806 are located near a center area of the brush head 802, while the bristles 808 configured to emit the electromagnetic radiation of the second wavelength 810 are located at a peripheral area of the brush head 802. In one example, the electromagnetic radiation of the first and the second wavelengths 806, 810 are coupled separately to the two sets of bristles 804, 808, such that the electromagnetic radiation of the first wavelength 806 is only coupled to the shorter bristles located near the center area 804, and the electromagnetic radiation of the second wavelength 810 is only coupled to the longer bristles located at the peripheral area 808. In another example, the brush head 802 is an oscillating brush head configured to rotate around an axis that is parallel to the Z direction.

The lengths of the bristles 804, 808 and the particular arrangement of the bristles 804, 808 may facilitate oral treatment procedures. For example, the lengths and the arrangements of the bristles 804, 808 depicted at 800 and 820 may enable simultaneous teeth whitening (i.e., using the electromagnetic radiation of the first wavelength 806) and treatment of the gums (i.e., using the electromagnetic radiation of the second wavelength 810). The simultaneous teeth whitening and gum treatment is depicted at 860. At 860, a surface of a tooth 862 receives a whitening treatment through its exposure to the electromagnetic radiation of the first wavelength 806. At the same time, a gums portion of a mouth 864 receives treatment through its exposure to the electromagnetic radiation of the second wavelength 810.

The oral treatment device 840 of FIG. 8 is similar to the device 800 and includes the bristles 804, 808 configured to emit the electromagnetic radiation of the first and the second wavelengths 806, 810, respectively. The oral treatment device 840 differs from the device 800 because the bristles 804 configured to emit the electromagnetic radiation of the first wavelength 806 have tips 842 that are of a cone shape or a double-beveled shape. The cone shape or the double-beveled shape of the tips 842 causes the electromagnetic radiation 806 to leave the tips 842 at an angle, such that the emitted light has a peak concentration along a direction that is not parallel to longitudinal axes of the bristles 804. In this example, the bristles 808 are configured to be substantially opaque to the electromagnetic radiation of the first wavelength 806, such that the bristles 808 act as light-shielding bristles to the output of the first plurality of bristles 804. As described above with respect to the devices 600, 640 of FIG. 6, the use of light-shielding bristles surrounding light-emitting bristles may be used to effectively confine the light emitted by the light-emitting bristles to a volume of space within the light-shielding bristles. Similarly, in the oral treatment device 800 of FIG. 8, the bristles 808 used for the second wavelength of light 810 may be substantially opaque to the electromagnetic radiation of the first wavelength 806, and the bristles 804 used for the first wavelength of light 806 may be substantially opaque to the electromagnetic radiation of the second wavelength 810.

While the disclosure has been described in detail and with reference to specific embodiments thereof, it will be apparent to one skilled in the art that various changes and modifications can be made therein without departing from the spirit and scope of the embodiments. Thus, it is intended that the present disclosure cover the modifications and variations of this disclosure provided they come within the scope of the appended claims and their equivalents.

It should be understood that as used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise. Further, as used in the description herein and throughout the claims that follow, the meaning of “each” does not require “each and every” unless the context clearly dictates otherwise. Finally, as used in the description herein and throughout the claims that follow, the meanings of “and” and “or” include both the conjunctive and disjunctive and may be used interchangeably unless the context expressly dictates otherwise; the phrase “exclusive of” may be used to indicate situations where only the disjunctive meaning may apply.

It is claimed:

1. A tissue treatment device comprising:
a bristle having a longitudinal axis, the bristle being configured to emit a peak concentration of electromagnetic radiation along a direction that is not parallel to the longitudinal axis;
a brush head for holding the bristle, the bristle projecting outwardly from the brush head and being configured to brush a surface; and
an electromagnetic energy source optically coupled to the bristle, the bristle being a waveguide configured to receive electromagnetic radiation from the electromagnetic energy source at a coupling point and to emit the received electromagnetic radiation at a tip.

2. The tissue treatment device of claim 1, wherein the coupling point has a shape configured to cause the emission of the electromagnetic radiation along the direction that is not parallel to the longitudinal axis.

3. The tissue treatment device of claim 2, wherein the coupling point has a beveled shape, a double-beveled shape, or a cone shape, and wherein the peak concentration of the electromagnetic radiation is emitted in a ring pattern, a diameter of the ring pattern increasing as a distance from the tip increases.

4. The tissue treatment device of claim 1, wherein the tip has a shape configured to cause the emission of the electromagnetic radiation along the direction that is not parallel to the longitudinal axis.

5. The tissue treatment device of claim 4, wherein the tip has a cone shape, and wherein the peak concentration of the electromagnetic radiation is emitted in a ring pattern, a diameter of the ring pattern increasing as a distance from the tip increases.

6. The tissue treatment device of claim 4, wherein the tip has a double-beveled shape, and wherein the peak concentration of the electromagnetic radiation is emitted in a pattern having opposing lobes, a size of the opposing lobes increasing as a distance from the tip increases.

7. The tissue treatment device of claim 1, further comprising:
a plurality of the bristles, wherein the plurality of the bristles have tips with beveling on a single side, the beveling being configured to cause the emitted electromagnetic radiation to be in a direction that is substantially perpendicular to a direction that is parallel to the longitudinal axes, and wherein the direction is towards a center area of the brush head.

8. The tissue treatment device of claim 1, wherein the tip has surface roughness features, the surface roughness features being configured to diffuse the electromagnetic radiation emitted from the tip in a plurality of directions.

9. The tissue treatment device of claim 8, wherein the tip is made of plastic having an index of refraction of approximately 1.4, and wherein the surface roughness features are configured to diffuse the electromagnetic radiation emitted from the tip in the plurality of directions when the tip is in an air environment, the air environment having an index of refraction of approximately 1.0.

10. The tissue treatment device of claim 8, wherein the surface roughness features have sizes in a range of approximately 5 μm to 50 μm.

11. The tissue treatment device of claim 8, wherein the surface roughness features are generated by an injection molding process, a sandblasting process, or a beadblasting process.

12. The tissue treatment device of claim 8, wherein the surface roughness features are configured to transfer a substantial portion of the electromagnetic radiation emitted from the tip to a surface or a substance that is in contact with the tip.

13. The tissue treatment device of claim 12, wherein the tip is made of plastic having an index of refraction of approximately 1.4, and wherein the surface roughness features are configured to transfer the substantial portion of the electromagnetic radiation emitted from the tip to the surface or the substance that is in contact with the tip when the surface or the substance has an index of refraction of approximately 1.3.

14. The tissue treatment device of claim 1, wherein the bristle is surrounded by a plurality of light-shielding bristles, the light-shielding bristles being substantially opaque to the electromagnetic radiation emitted by the bristle.

15. The tissue treatment device of claim 14, wherein the plurality of light-shielding bristles are located at a peripheral area of the brush head, wherein the bristle is located at a center area of the brush head, and wherein the tip of the bristle has a cone shape or a double-beveled shape configured to cause the emission of the electromagnetic radiation along the direction that is not parallel to the longitudinal axes.

16. A tissue treatment device comprising:
an electromagnetic energy source configured to generate electromagnetic radiation of a wavelength;
an optical lens configured to focus the electromagnetic radiation at a focal point that is a distance from the optical lens;
a plurality of light-shielding bristles having lengths that are longer than the distance, the light-shielding bristles being substantially opaque to the electromagnetic radiation; and
a brush head for holding the optical lens and the light-shielding bristles, the light-shielding bristles surrounding the optical lens on the brush head and projecting outwardly from the brush head in a direction substantially perpendicular to the brush head.

17. The tissue treatment device of claim 16, wherein the plurality of light-shielding bristles are located at a peripheral area of the brush head, and wherein the optical lens is located at a center area of the brush head.

18. The tissue treatment device of claim 16, wherein the electromagnetic energy source is configured to generate electromagnetic radiation of a second wavelength, and wherein the electromagnetic radiation of the second wavelength is emitted by one or more of the plurality of the light-shielding bristles.

19. An tissue treatment device comprising:
a first bristle configured to emit electromagnetic radiation of a first wavelength;
a second bristle configured to emit electromagnetic radiation of a second wavelength;
a brush head for holding the first and the second bristles, the bristles projecting outwardly from the brush head and being configured to brush a surface; and
an electromagnetic energy source optically coupled to the first and the second bristles, the electromagnetic energy source being configured to generate the electromagnetic radiation of the first and the second wavelengths.

20. The tissue treatment device of claim 19, wherein the electromagnetic radiation of the first wavelength is configured to whiten teeth, and wherein the electromagnetic radiation of the second wavelength is configured to treat gums of a mouth.

21. The tissue treatment device of claim 19, further comprising:
a plurality of the first bristles;
a plurality of the second bristles, wherein a length of the first bristle is less than a length of the second bristle, and wherein the plurality of the first bristles is located at a center area of the brush head, and the plurality of the second bristles is located at a peripheral area of the brush head.

22. The tissue treatment device of claim 21, wherein the length of the first bristle is within a range of approximately 2 mm-4 mm, and wherein the length of the second bristle is within a range of approximately 5 mm-8 mm.

23. The tissue treatment device of claim 21, wherein the electromagnetic radiation of the first wavelength is only coupled to the plurality of the first bristles, and wherein the electromagnetic radiation of the second wavelength is only coupled to the plurality of the second bristles.

24. The tissue treatment device of claim 21, wherein the second bristle is substantially opaque to the electromagnetic radiation of the first wavelength, and wherein the first bristle is substantially opaque to the electromagnetic radiation of the second wavelength.

25. The tissue treatment device of claim 24, wherein the first bristle emits the electromagnetic radiation via a tip that has a cone shape or a double-beveled shape, and wherein the cone shape or the double-beveled shape is configured to cause the emitted electromagnetic radiation to have a peak concentration along a direction that is not parallel to a longitudinal axis of the first bristle.

26. The tissue treatment device of claim 19, wherein the first wavelength is within a range of approximately 390 nm to 480 nm, and wherein the second wavelength is within a range of approximately 620 nm to 680 nm.

27. The tissue treatment device of claim 26, wherein the first bristle is made of polyethylene or polycarbonate, and wherein the second bristle is made of red-tinted transparent polyethylene or a material that is transparent to the second wavelength and that has a coating that is reflective or opaque to the first wavelength.
